(19) United States
(12) Patent Application Publication
(10) Pub. No.: US 2023/0200654 A1
(43) Pub. Date: Jun. 29, 2023

(54) ELECTRONIC DEVICE

(71) Applicant: InnoLux Corporation, Miao-Li County (TW)

(72) Inventors: Yuan-Lin WU, Miao-Li County (TW); Kuan-Feng LEE, Miao-Li County (TW)

(73) Assignee: InnoLux Corporation, Miao-Li County (TW)

(21) Appl. No.: 18/070,436

(22) Filed: Nov. 28, 2022

(30) Foreign Application Priority Data
Dec. 29, 2021 (CN) 202111639673.8

(51) Int. Cl. A61B 5/00; H05K 1/02

(52) U.S. Cl. CPC A61B 5/0059; H05K 1/0296; H05K 2201/10151; H05K 1/028; A61B 2562/04; A61B 2562/185

(57) ABSTRACT

An electronic device is provided by the present disclosure. The electronic device includes a patterned substrate having a plurality of main portions and a plurality of connecting portions, wherein at least one of the plurality of connecting portions connects two adjacent ones of the plurality of main portions, a plurality of biosensors disposed corresponding to the plurality of main portions, a conductive line disposed on the at least one of the plurality of connecting portions and electrically connecting two adjacent ones of the plurality of biosensors, and an insulating layer disposed on the plurality of biosensors and the conductive line.

### BACKGROUND OF THE DISCLOSURE

### 1. Field of the Disclosure

The present disclosure relates to an electronic device, and more particularly to a stretchable electronic device.

### 2. Description of the Prior Art

Biosensors can detect the physiological signals of the human body and convert them into electronic signals to obtain the physiological information of the subject. In order to improve the reusability of biosensors or the adaptability of biosensors in the using environment, the development of stretchable biosensors is still an important issue in this field.

### SUMMARY OF THE DISCLOSURE

In some embodiments, an electronic device is provided by the present disclosure. The electronic device includes a patterned substrate, a plurality of biosensors, a conductive line and an insulating layer. The patterned substrate has a plurality of main portions and a plurality of connecting portions, wherein at least one of the plurality of connecting portions connects two adjacent ones of the plurality of main portions. The plurality of biosensors are respectively disposed corresponding to one of the plurality of main portions. The conductive line is disposed on at least one of the plurality of connecting portions and electrically connects two adjacent ones of the plurality of biosensors. The insulating layer is disposed on the plurality of biosensors and the conductive line.

These and other objectives of the present disclosure will no doubt become obvious to those of ordinary skill in the art after reading the following detailed description of the embodiment that is illustrated in the various figures and drawings.

### BRIEF DESCRIPTION OF THE DRAWINGS

FIG. 1 schematically illustrates a top view of an electronic device according to a first embodiment of the present disclosure.

FIG. 2 schematically illustrates a partial cross-sectional view of the electronic device according to the first embodiment of the present disclosure.

FIG. 3 schematically illustrates a cross-sectional view of an electronic device according to a second embodiment of the present disclosure.

FIG. 4 schematically illustrates a cross-sectional view of an electronic device according to a third embodiment of the present disclosure.

FIG. 5 schematically illustrates a cross-sectional view of an electronic device according to a fourth embodiment of the present disclosure.

FIG. 6 schematically illustrates a cross-sectional view of the electronic device shown in FIG. 1 along a cutting line G-G′.

FIG. 7 schematically illustrates a top view of a conductive line of an electronic device according to a fifth embodiment of the present disclosure.

FIG. 8 schematically illustrates a top view of an electronic device according to a sixth embodiment of the present disclosure.

FIG. 9 schematically illustrates an application of an electronic device according to a seventh embodiment of the present disclosure.

FIG. 10 schematically illustrates the manufacturing process of an electronic device according to an eighth embodiment of the present disclosure.

FIG. 11 schematically illustrates the manufacturing process of an electronic device according to a ninth embodiment of the present disclosure.

### DETAILED DESCRIPTION

The present disclosure may be understood by reference to the following detailed description, taken in conjunction with the drawings as described below. It is noted that, for purposes of illustrative clarity and being easily understood by the readers, various drawings of this disclosure show a portion of the electronic device, and certain elements in various drawings may not be drawn to scale. In addition, the number and dimension of each element shown in drawings are only illustrative and are not intended to limit the scope of the present disclosure.

Certain terms are used throughout the description and following claims to refer to particular elements. As one skilled in the art will understand, electronic equipment manufacturers may refer to an element by different names. This document does not intend to distinguish between elements that differ in name but not function.

In the following description and in the claims, the terms “include”, “comprise” and “have” are used in an open-ended fashion, and thus should be interpreted to mean “include, but not limited to . . . ”.

It will be understood that when an element or layer is referred to as being “disposed on” or “connected to” another element or layer, it can be directly on or directly connected to the other element or layer, or intervening elements or layers may be presented (indirectly). In contrast, when an element is referred to as being “directly on” or “directly connected to” another element or layer, there are no intervening elements or layers presented. When an element or a layer is referred to as being “electrically connected” to another element or layer, it can be a direct electrical connection or an indirect electrical connection. The electrical connection or coupling described in the present disclosure may refer to a direct connection or an indirect connection. In the case of a direct connection, the ends of the elements on two circuits are directly connected or connected to each other by a conductor segment. In the case of an indirect connection, switches, diodes, capacitors, inductors, resistors, other suitable elements or combinations of the above elements may be included between the ends of the elements on two circuits, but not limited thereto.

Although terms such as first, second, third, etc., maybe used to describe diverse constituent elements, such constituent elements are not limited by the terms. The terms are used only to discriminate a constituent element from other constituent elements in the specification. The claims may not use the same terms, but instead may use the terms first, second, third, etc. with respect to the order in which an element is claimed. Accordingly, in the following description, a first constituent element maybe a second constituent element in a claim.

According to the present disclosure, the thickness, length and width may be measured through optical microscope, and the thickness or width may be measured through the cross-sectional view in the electron microscope, but not limited thereto.

In addition, any two values or directions used for comparison may have certain errors. In addition, the terms “equal to”, “equal”, “the same”, “approximately” or “substantially” are generally interpreted as being within ±20%, ±10%, ±5%, ±3%, ±2%, ±1%, or ±0.5% of the given value.

In addition, the terms “the given range is from a first value to a second value” or “the given range is located between a first value to a second value” represents that the given range includes the first value, the second value and other values there between.

If a first direction is said to be perpendicular to a second direction, the included angle between the first direction and the second direction maybe located between 80 to 100 degrees. If a first direction is said to be parallel to a second direction, the included angle between the first direction and the second direction may be located between 0 to 10 degrees.

Unless it is additionally defined, all terms (including technical and scientific terms) used herein have the same meaning as commonly understood by those ordinary skilled in the art. It can be understood that these terms that are defined in commonly used dictionaries should be interpreted as having meanings consistent with the relevant art and the background or content of the present disclosure, and should not be interpreted in an idealized or overly formal manner, unless it is specifically defined in the embodiments of the present disclosure.

In the present disclosure, the electronic device may include a display device, a sensing device, a back-light device, an antenna device or a tiled device, but not limited thereto. The electronic device may be a foldable electronic device, a flexible electronic device or a stretchable electronic device. For example, the electronic device of the present disclosure may include a biometric sensing device. The biometric sensing device may for example include a photoelectric biosensor, a piezoelectric biosensor, other suitable types of biosensors or combinations of the above-mentioned biosensors, but not limited thereto. The biosensor may be the combination of an emitting source and a receiving source or include the structure that can self-emit and self-receive. The photoelectric biosensor may for example include a photo sensor and/or a light emitting unit, wherein the photo sensor may include photodiodes, and the light emitting unit may include light emitting diodes, but not limited thereto. The photodiode may include an organic photodiode, but not limited thereto. The light emitting diode may for example include an organic light emitting diode (OLED), a mini light emitting diode (mini LED), a micro light emitting diode (micro LED) or a quantum dot light emitting diode (QLED), but not limited thereto. The piezoelectric biosensor may include sensors using piezoelectric materials, wherein the piezoelectric materials may include polyvinylidene fluoride (PVDF), but not limited thereto. The biosensors may detect the physiological signals through optical sensing or pressure sensing. For example, the biosensors may be used to detect fingerprint, electroencephalogram (EEG), electrooculogram (EOG), electromyogram (EMG), electrocardiogram (ECG), airflow, respiration efforts, oxygen saturation or other physiological signals. In terms of applications, the photoelectric biosensor may for example be used to detect wound healing, blood oxygen concentration, etc., while the piezoelectric biosensor may for example be used to monitor breathing, heartbeat or sleep quality, but not limited thereto.

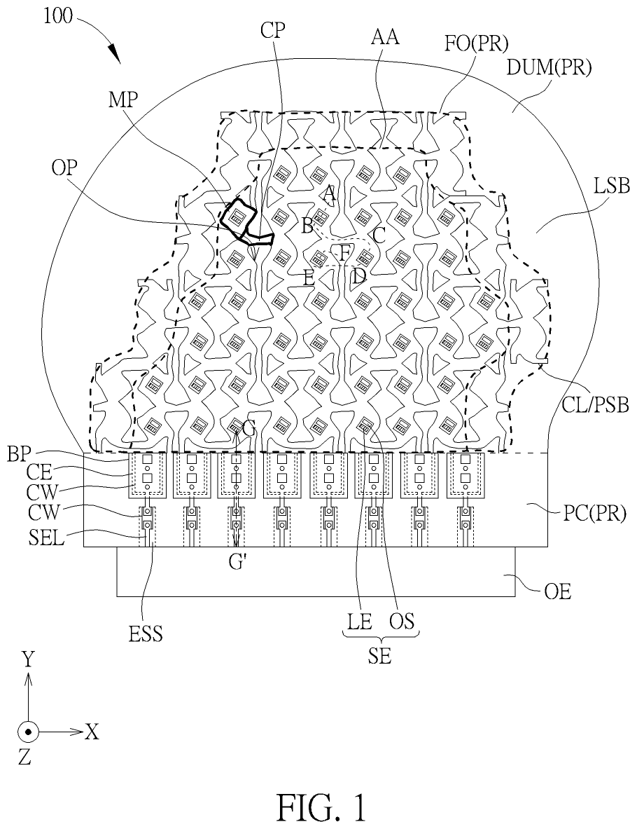

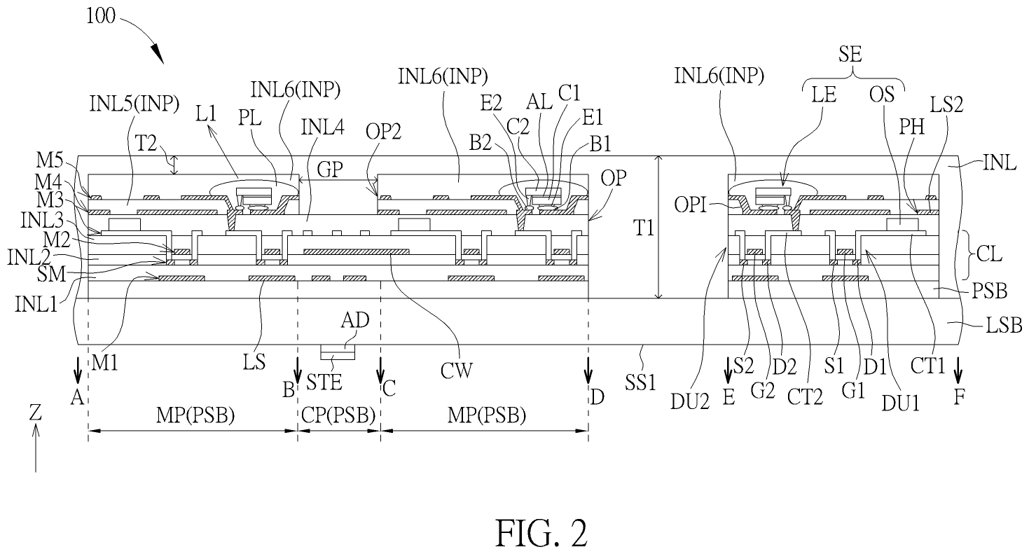

Referring to FIG. 1 and FIG. 2, FIG. 1 schematically illustrates a top view of an electronic device according to a first embodiment of the present disclosure, and FIG. 2 schematically illustrates a partial cross-sectional view of the electronic device according to the first embodiment of the present disclosure. Specifically, FIG. 2 schematically illustrates a cross-sectional view of the electronic device shown in FIG. 1 along a line segment ABCDEF. According to the present embodiment, the electronic device 100 may include a supporting substrate LSB, a patterned substrate PSB, a circuit layer CL, biosensors SE and an insulating layer INL, wherein the patterned substrate PSB is disposed on the supporting substrate LSB, the circuit layer CL is disposed on the patterned substrate PSB, the biosensors SE are disposed on the circuit layer CL, and the insulating layer INL is disposed on the biosensors SE and the circuit layer CL and covers the biosensors SE and the circuit layer CL, but not limited thereto. The elements and/or layers included in the electronic device 100 will be detailed in the following.

The supporting substrate LSB may be disposed under the patterned substrate PSB. According to the present embodiment, the supporting substrate LSB may include a flexible substrate, wherein the term “flexible” represents that it can be curved, folded, rolled, stretched or deformed in other ways. For example, the supporting substrate LSB maybe a stretchable substrate, but not limited thereto. The supporting substrate LSB may be used to support the layers and/or structures disposed thereon. It should be noted that although the supporting substrate LSB is shown as a single layer in FIG. 2, the present embodiment is not limited thereto. In some embodiments, the supporting substrate LSB may include a multi-layer structure. In addition, the supporting substrate LSB may include biocompatible materials in the present embodiment, that is, the supporting substrate LSB may be a biocompatible substrate, but not limited thereto. For example, the supporting substrate LSB may include organic materials, but not limited thereto. Since the electronic device 100 of the present embodiment may serve as the biosensor, the electronic device 100 maybe in contact with the subject (for example, in contact with the skin of the subject). In this case, since the supporting substrate LSB includes the biocompatible materials, the possibility of adverse effects of the supporting substrate LSB on the subject may be reduced.

The patterned substrate PSB may be disposed on the supporting substrate LSB. For example, although it is not shown in FIG. 2, the supporting substrate LSB may be adhered to a surface of the patterned substrate PSB through an adhesive layer, but not limited thereto. According to the present embodiment, the patterned substrate PSB may include a flexible substrate or partially be a flexible substrate. For example, the patterned substrate PSB may be a stretchable substrate, but not limited thereto. The material of the flexible substrate may include polyimide (PI), polycarbonate (PC), polyethylene terephthalate (PET), other suitable materials or combinations of the above-mentioned materials. It should be noted that although the patterned substrate PSB is shown as a single layer in FIG. 2, the present embodiment is not limited thereto. In some embodiments, the patterned substrate PSB may include a multi-layer structure, wherein the multi-layer structure may include an inorganic insulating layer (such as silicon oxide (SiOₓ)) to improve water and oxygen blocking effect of the patterned substrate PSB.

According to the present embodiment, the patterned substrate PSB may include a plurality of main portions MP and a plurality of connecting portions CP, wherein at least one of the connecting portions CP may connect adjacent two of the main portions MP. Specifically, as shown in FIG. 1 and FIG. 2, each of the main portions MP of the patterned substrate PSB may be connected to at least one of the connecting portions CP and connected to other main portions MP through the connecting portion CP to which it is connected. As shown in FIG. 1, the main portion MP may for example be island-shaped, and the connecting portion CP may for example be string-shaped in the present embodiment, but not limited thereto. In some embodiments, the shape of the main portion MP maybe a rectangle, a circle or other suitable shapes. The main portion MP may be configured to dispose the biosensor SE or other active elements (such as the channel region of the thin film transistors in the circuit layer, but not limited thereto) thereon. In other words, the biosensors SE and/or the active elements in the circuit layer CL may be disposed corresponding to the main portions MP. It should be noted that “the biosensors SE and/or the active elements in the circuit layer CL may be disposed corresponding to the main portions MP” mentioned above may represent that the biosensors SE and the active elements are overlapped with or at least partially overlapped with the main portions MP in a top view direction (such as the direction Z) of the electronic device 100, but not limited thereto. In the embodiment shown in FIG. 2, the biosensors SE may be completely overlapped with the main portions MP in the direction Z, but the present disclosure is not limited thereto. The definition of the term “corresponding to” mentioned above may be applied to each of the embodiments of the present disclosure, which will not be redundantly described. The connecting portion CP may change the distance between the adjacent main potions MP to which it is connected. For example, when the electronic device 100 is deformed (such as being stretched), the connecting portions CP may be deformed, and the sizes (such as length) of the connecting portions CP may be changed due to deformation, thereby changing the distance between the main portions MP. Or, the connecting portions CP with different sizes may be designed through different pattern designs, thereby changing the distances between the adjacent main portions MP. In addition, a portion of the circuit layer CL may be disposed on the connecting portions CP, and a portion of the wires and/or the elements in the circuit layer CL may be disposed corresponding to the connecting portions CP, but not limited thereto. The patterned substrate PSB of the present embodiment may for example be formed by forming openings OP in a substrate. Specifically, a complete substrate may be formed on the supporting substrate LSB at first, and a plurality of openings OP maybe formed in the subsequent processes, wherein the openings OP may penetrate through the complete substrate and expose the supporting substrate LSB, thereby forming the patterned substrate PSB. It should be noted that the pattern of the patterned substrate PSB shown in FIG. 1 is just exemplary, and the present disclosure is not limited thereto. According to the demands of the product, the main portions MP and the connecting portions CP may respectively include any suitable shape and arrangement, so as to form the patterned substrates PSB with different patterns.

The circuit layer CL maybe disposed on the patterned substrate PSB, and the circuit layer CL maybe patterned according to the shape of the patterned substrate PSB. In other words, the pattern of the circuit layer CL and the pattern of the patterned substrate PSB may be the same. In the present embodiment, a complete circuit layer CL may be disposed on the patterned substrate PSB which is not yet patterned at first, and the circuit layer CL may be penetrated when forming the openings OP shown in FIG. 1 and FIG. 2 in the subsequent processes to form the patterned circuit layer CL, but not limited thereto.

According to the present embodiment, the circuit layer CL may include any wire, circuit, active element and/or passive element that can be applied to the electronic device 100. For example, as shown in FIG. 2, the circuit layer CL may include driving units DU1 and driving units DU2 which are electrically connected to the biosensors SE, wherein the driving units DU1 may be electrically connected to the photo sensors OS in the biosensors SE, thereby controlling the on/off of the photo sensors OS to which they are electrically connected, and the driving units DU2 may be electrically connected to the light emitting units LE in the biosensors SE, thereby controlling the light emission of the light emitting units LE to which they are electrically connected, but not limited thereto. Specifically, the driving unit DU1 and the driving unit DU2 may for example include thin film transistor respectively, wherein the photo sensor OS may for example be electrically connected to the drain D1 of the driving unit DU1 through a connecting element CT1, and the light emitting unit LE may for example be electrically connected to the drain D2 of the driving unit DU2 through a connecting element CT2.

As shown in FIG. 2, the circuit layer CL of the present embodiment may include a metal layer M1, a semiconductor layer SM, a metal layer M2 and a metal layer M3, wherein the semiconductor layer SM may form the channel region, the source S1 and the drain D1 of the driving unit DU1 and the channel region, the source S2 and the drain D2 of the driving unit DU2, and the metal layer M2 may form the gate G1 of the driving unit DU1 and the gate G2 of the driving unit DU2, but not limited thereto. The channel regions of the driving unit DU1 and the driving unit DU2 may respectively be defined as a portion of the semiconductor layer SM overlapped with the gate G1 and overlapped with the gate G2. The metal layer M3 may form the connecting element CT1 and the connecting element CT2. The metal layer M1 may be optionally disposed. In some embodiments, the metal layer M1 may form light shielding elements LS disposed under the driving units DU1 and the driving units DU2, that is, the light shielding elements LS may be disposed corresponding to the driving units DU1 and/or the driving units DU2. Further, the light shielding elements LS corresponding to the driving units DU1 and the driving units DU2 maybe regarded as being disposed corresponding to the main portions MP, but not limited thereto. In some embodiments, the metal layer M1 maybe used to form the gates of the driving units DU1 and/or the driving units DU2. Since the light shielding elements LS may be disposed under the driving units DU1 and/or the driving units DU2, the influence of the light (such as ambient light) on the driving units DU1 and the driving units DU2 may be reduced. The metal layer M1, the metal layer M2 and the metal layer M3 may include any suitable conductive material, such as metal materials, but not limited thereto. The material of the semiconductor layer SM may for example include low temperature polysilicon (LTPS), low temperature polysilicon oxide (LTPO) or amorphous silicon, but not limited thereto. It should be noted that although the driving units DU1 and/or the driving units DU2 shown in FIG. 2 are top gate thin film transistors, the present disclosure is not limited thereto. In other embodiments, the driving unit DU1 and/or the driving unit DU2 may include bottom gate thin film transistors or double gate (or dual gate) thin film transistors. As shown in FIG. 2, the circuit layer CL may further include an insulating layer INL1 located between the metal layer M1 and the semiconductor layer SM, an insulating layer INL2 located between the semiconductor layer SM and the metal layer M2 and an insulating layer INL3 covering the metal layer M2, wherein the insulating layer INL1, the insulating layer INL2 and the insulating layer INL3 may include any suitable insulating material. It should be noted that the numbers of the metal layers and the insulating layers in the circuit layer CL and the disposition ways of the electronic elements shown in FIG. 2 are just exemplary, and the present disclosure is not limited thereto. As mentioned above, the active elements (such as the driving unit DU1 and the driving unit DU2) of the circuit layer CL may be disposed corresponding to the main portions MP of the patterned substrate PSB in the present embodiment, but not limited thereto. In addition, although it is not shown in FIG. 2, a portion of the elements of the driving unit DU1 and/or the driving unit DU2 (such as the source/drain) may be disposed on the connecting portion CP or disposed corresponding to the connecting portion CP.

The circuit layer CL of the present embodiment may further include at least one conductive line CW in addition to the above-mentioned elements. In the present embodiment, the conductive line CW may represent the signal lines electrically connected to the driving units DU1 and/or the driving units DU2, other suitable wires in the circuit layer CL, or combinations of the above-mentioned elements, but not limited thereto. According to the present embodiment, the conductive line CW may be disposed on at least one connecting portion CP of the patterned substrate PSB, and the conductive wire CW may electrically connect two adjacent biosensors SE. In detail, since the connecting portion CP may connect two adjacent main portions MP, and the conductive line CW may be disposed on the connecting portion CP, the two ends of the conductive line CW may respectively extend to the two adjacent main portions MP and be electrically connected to the biosensors SE respectively on the two adjacent main portions MP, such that the two biosensors SE located on different main portions MP may be electrically connected to each other through the conductive line CW located on the connecting portion CP. Specifically, the conductive line CW may be electrically connected to the photo sensor OS by electrically connecting to the driving unit DU1, or the conductive line CW may be electrically connected to the light emitting unit LE by electrically connecting to the driving unit DU2. It should be noted that “the conductive line CW is electrically connected to the biosensor SE” mentioned above may include the condition that the conductive line CW is electrically connected to the photo sensor OS or the light emitting unit LE, but the present disclosure is not limited thereto.

According to the present embodiment, in the process of electrically connecting two adjacent biosensors SE, the conductive line CW does not need to be transferred to another layer, that is, the conductive line CW is formed of the same metal layer. As shown in FIG. 2, the conductive line CW may for example be electrically connected to the photo sensor OS and/or the light emitting unit LE by electrically connecting to the gates of the driving unit DU1 and/or the driving unit DU2, and the conductive line CW and the gates of the driving unit DU1 and/or the driving unit DU2 may be formed of the same metal layer M2, but not limited thereto.

According to the present embodiment, since the conductive line CW may be electrically connected to different biosensors SE, when the conductive line CW serve as the signal lines for transmitting sensing signals and/or light emitting signals, the number of the conductive lines CW in the circuit layer CL may be reduced, thereby simplifying the layout of the wires or reducing the size of the electronic device 100. The material of the conductive line CW may for example be the same as the materials of the metal layer M1, the semiconductor layer SM and the metal layer M2, and will not be redundantly described. It should be noted that the elements and/or wires included in the circuit layer CL of the present embodiment are not limited to the above-mentioned contents, and other suitable electronic elements and/or wires maybe included in the circuit layer CL.

The biosensor SE of the present embodiment may include the light emitting unit(s) LE and the photo sensor(s) OS, wherein the light emitting unit LE and the photo sensor OS may be disposed corresponding to the main portion MP. For example, a biosensor SE may for example be disposed corresponding to a main portion MP. Therefore, a main portion MP may correspond to a light emitting unit LE and a photo sensor OS, but not limited thereto. In some other embodiments, more than one biosensors SE may be disposed on a main portion MP. As shown in FIG. 2, taking a biosensor SE as an example, the light emitting unit(s) LE included in the biosensor SE may emit a light L1, wherein the light L1 may be reflected by the subject and be detected by the photo sensor(s) OS in the biosensor SE, thereby obtaining the physiological information. That is, the biosensor SE of the present embodiment may for example be a photoelectric biosensor, but not limited thereto. In some embodiments, when the electronic device does not have the function of light sensing or light recognition, the biosensor SE may not include the light emitting unit LE. In some embodiments, the light emitting unit LE may provide the display function of the electronic device 100 in addition to emitting light for detection (such as the light L1). For example, the light emitting unit LE may be time-sharing controlled, such that the light emitting unit LE may emit the light source for detection in a time period and emit the light source for display in another time period to display images, but not limited thereto.

According to the present embodiment, the biosensors SE and the circuit layer CL (for example, the conductive line CW) may be disposed at the same side of the patterned substrate PSB. It should be noted that “the same side of the patterned substrate PSB” mentioned here may be the same side of the patterned substrate PSB in a space, which is not limited to a plane. Specifically, by disposing the biosensors SE and the conductive line CW at the same side of the patterned substrate PSB, the disposition of the layers may be adjusted more easily, such that the biosensors SE and/or the circuit layer CL may be located at the neutral axis of the electronic device 100, thereby reducing the influence of stress on the biosensors SE and/or the circuit layer CL. Therefore, the possibility of damage to the wires or the elements of the biosensors SE and/or the circuit layer CL may be reduced. In addition, since the biosensors SE and/or the circuit layer CL may be located at the same side of the patterned substrate PSB, the situation that the stress difference between the biosensors SE and the circuit layer CL is too great due to the excessive great distance between the biosensors SE and the circuit layer CL may be reduced.

As shown in FIG. 2, the electronic device 100 may further include an insulating layer INL4 disposed on the photo sensors OS, a metal layer M4 disposed on the insulating layer INL4, an insulating layer INL5 disposed on the metal layer M4 and a metal layer M5 disposed on the insulating layer INL5, but not limited thereto. The insulating layer INL4, the insulating layer INL5, the metal layer M4 and the metal layer M5 may be disposed corresponding to the main portions MP of the patterned substrate PSB, but not limited thereto. According to the present embodiment, the light emitting unit LE may be electrically connected to the driving unit DU2 or other electronic elements through the metal layer M4 and the metal layer M5. For example, the light emitting unit LE may for example be an inorganic light emitting diode in the present embodiment and include a semiconductor layer C1, a semiconductor layer C2, an active layer AL disposed between the semiconductor layer C1 and the semiconductor layer C2, an electrode E1 connected to the semiconductor layer C1 and an electrode E2 connected to the semiconductor layer C2, but not limited thereto. As shown in FIG. 2, the electrode E1 and the electrode E2 of the light emitting unit LE may be electrically connected to the metal layer M5 and/or the metal layer M4 respectively through a bonding material B1 and a bonding material B2, such that the light emitting unit LE may be electrically connected to the driving unit DU2 or other electronic elements. The bonding material B1 and the bonding material B2 may for example include anisotropic conductive film (ACF), tin (Sn), gold-tin alloy, silver glue, other suitable materials or combinations of the above-mentioned materials, but not limited thereto. The insulating layer INL5 may provide the function of defining the light emitting region or defining the disposition position of the light emitting unit LE. For example, the insulating layer INL5 may include openings OPI, wherein each of the openings OPI is respectively located on each of the main portions MP, and each of the light emitting units LE may respectively be disposed in one of the openings OPI in the insulating layer INL5. In addition, the electronic device 100 may further include a protecting layer PL disposed on the light emitting units LE and an insulating layer INL6 covering the protecting layer PL, the metal layer M5 and the insulating layer INL5, wherein the insulating layer INL6 may be disposed corresponding to the main portions MP, but not limited thereto.

As shown in FIG. 2, the metal layer M4 disposed on the photo sensors OS of the biosensors SE may for example serve as a light shielding layer (for example, the metal layer M4 includes the light shielding element LS2) in the present embodiment, such that the influence of the non-detecting light (or the noise light) on the photo sensors OS may be reduced. That is, the electronic device 100 may further include light shielding elements LS2 formed of the metal layer M4 and disposed on the biosensors SE. In addition, the light shielding element LS2 may include a through hole PH, wherein the through hole PH may overlap the biosensor SE or overlap the photo sensor OS of the biosensor SE. Specifically, the metal layer M4 may be patterned to form a plurality of through holes PH, and each of the through holes PH may for example overlap a photo sensor OS respectively, but not limited thereto. By disposing the through holes PH that overlap the photo sensors OS in the light shielding elements LS2, the possibility that the detecting light (for example, the light L1) is blocked by the light shielding elements LS2 and affects the result of detection may be reduced. In addition, although it is not shown in FIG. 2, the insulating layer INL5 may include a light shielding material in other embodiments, and a portion of the insulating layer INL5 corresponding to the photo sensors OS may be removed to expose the photo sensors OS. The light shielding material may for example include black matrix, but not limited thereto. Since the insulating layer INL5 including the light shielding material would be disposed in the region not corresponding to the photo sensor OS, the noise light received by the photo sensors OS may be reduced, thereby improving the signal-to-noise ratio (S/N ratio).

According to the present embodiment, the electronic device 100 may include a plurality of insulating patterns INP disposed on the biosensors SE, wherein adjacent two of the insulating patterns INP may be spaced apart from each other through a gap GP, and the gap GP may overlap the connecting portion CP or correspond to the connecting portion CP. Specifically, as shown in FIG. 2, one of the insulating patterns INP may for example include a portion of the insulating layer INL6, but not limited thereto. In some embodiments, one of the insulating patterns INP may for example include a portion of the insulating layer INL6 and a portion of the insulating layer INL5. In the present embodiment, since the portion of the insulating layer INL6 and/or the insulating layer INL5 corresponding to the connecting portion CP may be removed, the gap GP may thereby be formed and separate the insulating layer INL6 and/or the insulating layer INL5 into the plurality of insulating patterns INP. That is, the insulating patterns INP may correspond to the main portions MP but not the connecting portions CP. The insulating patterns INP of the present embodiment may for example be formed by forming openings in the insulating layer. Specifically, a complete insulating layer INL6 and/or a complete insulating layer INL5 corresponding to the patterned substrate PSB may be formed at first, and then the portion of the insulating layer INL6 and/or the insulating layer INL5 corresponding to the connecting portions CP may be removed to form openings OP2, thereby forming the insulating patterns INP. Since the insulating pattern INP may not be included at the position corresponding to the connecting portions CP, the stress generated when the electronic device 100 is deformed (for example, being stretched) may be reduced, thereby improving durability or lifespan of the electronic device 100.

As shown in FIG. 2, the insulating layer INL may be disposed on the elements and/or the layers such as the biosensors SE, the conductive lines CW in the circuit layer CL, the driving elements (for example, the driving unit DU1 and the driving unit DU2), the patterned substrate PSB, and the like, but not limited thereto. That is, the insulating layer INL may be disposed on the insulating layer INL6 and filled into the opening(s) OP2 and the opening(s) OP. According to the present embodiment, the insulating layer INL may for example be an elastic cover layer used to separate the biosensors SE and the subject. Specifically, when the electronic device 100 is being used, the subject (for example, the skin of the subject) may be directly in contact with the insulating layer INL but not the biosensors SE, such that the possibility that the biosensor is damaged and the function thereof is affected may be reduced, or the adverse effects on the subject from electronic components such as biosensors SE may be reduced. In the present embodiment, the insulating layer INL may include biocompatible materials. For example, the insulating layer INL and the supporting substrate LSB may include the same material, but not limited thereto. Since the insulating layer INL may include biocompatible materials, the adverse effects on the subject from the insulating layer INL may be reduced.

According to the present embodiment, the thickness of the insulating layer INL corresponding to the main portions MP may be lower than the thickness of the insulating layer INL corresponding to the connecting portions CP. For example, as shown in FIG. 2, the portion of the insulting layer INL corresponding to the main portions MP may have a thickness T2, and the portion of the insulating layer INL corresponding to the connecting portions CP may have a thickness T1, wherein the thickness T1 may be greater than the thickness T2. When the electronic device 100 is being operated (for example, performing detection of physiological signals), the insulating layer INL may be in contact with the subject. Since the thickness T2 of the insulating layer INL corresponding to the main portions MP may be lower, when the subject is in contact with the insulating layer INL, the distance between the biosensors SE and the subject is closer, thereby improving the sensitivity or the accuracy of the biosensors SE. It should be noted that although FIG. 2 shows the structure that the upper surface of the insulating layer INL is flat, the present disclosure is not limited thereto. In some embodiments, the insulating layer INL may be conformally disposed on the insulating layer INL6 and/or the supporting substrate LSB, thereby forming a concave-convex upper surface, and the thickness difference in the entire insulating layer INL may for example be small. In this case, the thickness T2 of the portion of the insulating layer INL corresponding to the main portions MP may still be lower than the thickness T1 of the portion of the insulating layer INL corresponding to the connecting portions CP, thereby achieving the effect of improving the sensitivity or the accuracy of the biosensors SE, but not limited thereto. According to the present embodiment, the Young's module of the insulating layer INL may be lower than the Young's modules of the insulating layer INL1, the insulating layer INL2, the insulating layer INL3 and the insulating layer INL4. Therefore, even if the insulating layer INL is farther from the neutral axis, the insulating layer INL may be attached to the skin due to the lower Young's module, thereby reducing the possibility of breaking of the insulating layer. In addition, the light transmittance of the insulating layer INL may be greater than the light transmittance of the patterned substrate PSB and the light transmittance of the supporting substrate LSB in the present embodiment, thereby increasing the light transmittance or the signal-to-noise ratio of the biosensors SE. For example, the light transmittance of the insulating layer INL may be greater than 70%, but not limited thereto.

In the present embodiment, the electronic device 100 may optionally include stretch sensors STE, wherein the stretch sensors STE may be disposed corresponding to the connecting portions CP. Specifically, the stretch sensors STE may be adhered to the surface SS1 of the supporting substrate LSB away from the patterned substrate PSB, but not limited thereto. According to the present embodiment, the on/off of the biosensors SE may for example be controlled through the stretch sensors STE. When the connecting portion CP corresponding to a stretch sensor STE is stretched or deformed in other ways, the stretch sensor STE may detect the stretching signal and turn on the biosensor SE controlled by the stretch sensor STE. On the other hand, when the stretch sensor STE does not detect the stretching signal, the biosensor SE controlled by the stretch sensor STE may not be turned on, or the off-state of the biosensor SE may be maintained. In some embodiments, a stretch sensor STE may be used to control all of the biosensors SE in the electronic device 100. In some embodiments, a stretch sensor STE maybe used to control a portion of the biosensors SE in the electronic device 100. By controlling on/off of the biosensors SE through the stretch sensors STE, the power consumption of the biosensors SE may be reduced, thereby improving the performance of the electronic device 100.

As shown in FIG. 1, the electronic device 100 of the present embodiment may include an active region AA and a peripheral region PR. The active region AA may be the region of the electronic device 100 that includes the biosensors SE and used to detect physiological signals. According to the present embodiment, the active region AA may be defined through active elements such as biosensors SE, and the active region AA may for example be defined as the region enclosed by the outer edge of the outermost biosensors SE among the plurality of biosensors SE, and the region other than the active region AA may be defined as the peripheral region PR, but not limited thereto. In addition, the peripheral region PR may include a fan out region FO and a dummy region DUM in the present embodiment, but not limited thereto. The fan out region FO may include conductive lines, wires or other suitable electronic elements to pull the signal lines of the biosensor SE (such as the conductive line CW in FIG. 2) outward to be connected to external electronic elements, but not limited thereto. The dummy region DUM may be the region that includes the insulating layer INL and the supporting substrate LSB, but not limited thereto. In addition, the peripheral region PR of the electronic device 100 of the present embodiment may further include a peripheral circuit region PC, wherein at least one bonding pad BP may be disposed in the peripheral circuit region PC, and the signal lines (such as the conductive line CW) may be electrically connected to the bonding pad BP and to the external electronic element OE through the bonding pad BP. The external electronic element OE may for example include a flexible printed circuit board (FPCB), but not limited thereto. It should be noted that the ranges of the active region AA and the peripheral region PR marked in FIG. 1 are just exemplary, and do not represent the actual ranges of the active region AA and the peripheral region PR. In addition, the shapes of the active region AA and the peripheral region PR of the electronic device 100 are not limited to what is shown in FIG. 1, and the shapes of the active region AA and the peripheral region PR may be variable according to the design of the product.

Other embodiments of the present disclosure will be described in the following. In order to simplify the description, the same elements or layers in the following embodiments would be labeled with the same symbol, and the features thereof will not be redundantly described. The differences between the embodiments will be detailed in the following.

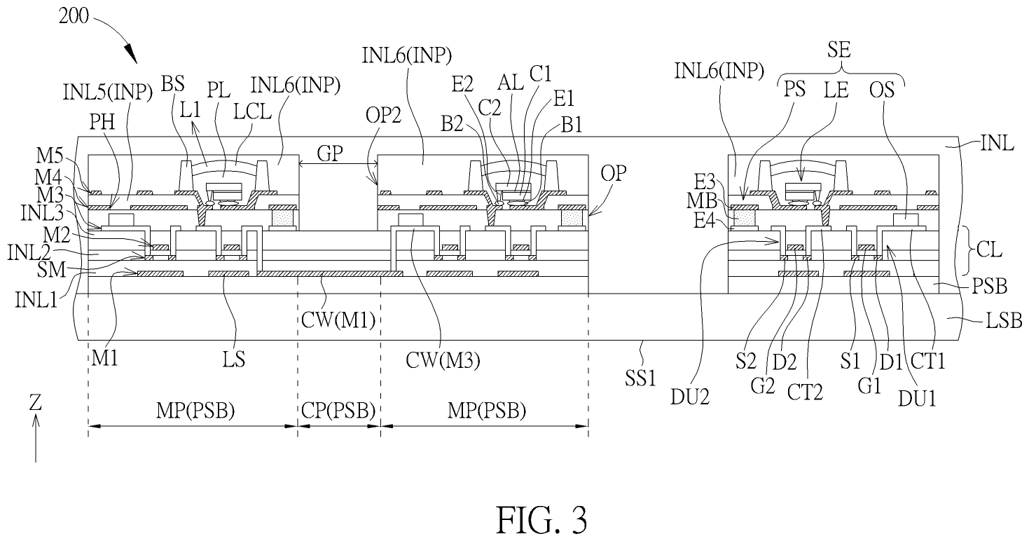

Referring to FIG. 3, FIG. 3 schematically illustrates a cross-sectional view of an electronic device according to a second embodiment of the present disclosure. According to the present embodiment, the electronic device 200 may include light shielding structures BS, wherein the light shielding structures BS may be disposed and surround the light emitting units LE. Specifically, the light shielding structures BS may be disposed on the metal layer M5 or the insulating layer INL5 and surround the light emitting units LE and/or the protecting layers PL, but not limited thereto. The light shielding structure BS may for example include a black matrix layer, but not limited thereto. By disposing the light shielding structures BS that surround the light emitting units LE in the electronic device 200, the proportion of the collimated light emitted by the light emitting units LE may be increased. Therefore, when the light emitting units LE serve as detecting light source, the accuracy or the sensitivity of the biosensors SE may be improved.

In the present embodiment, the electronic device 200 may include light converting layers LCL disposed on the light emitting units LE. Specifically, the light converting layers LCL may be disposed on the protecting layers PL and the light emitting units LE and be surrounded by the light shielding structures BS, but not limited thereto. The light converting layers LCL may convert the wavelength and/or color of the light emitted from the light emitting units LE. The light converting layer LCL may for example include fluorescence, phosphor, quantum dot, color filter, other suitable materials or combinations of the above-mentioned materials, but not limited thereto. The light converting layers LCL may convert the lights emitted from different light emitting units LE into the lights of the same color or different colors, according to the design of the product. For example, the light converting layers LCL may respectively convert the lights emitted from the light emitting units LE from left to right in FIG. 3 into green light, blue light and red light, but not limited thereto. In some embodiments, the electronic device 200 may include the light shielding structures BS and not include the light converting layer LCL.

In the present embodiment, the biosensors SE of the electronic device 200 may further include pressure sensors PS. That is, one of the biosensors SE may include at least one pressure sensor PS, at least one light emitting unit LE and at least one photo sensor OS, but not limited thereto. The pressure sensor(s) PS in a biosensor SE may be disposed on the main portion MP of the patterned substrate PSB in cooperation with the light emitting unit(s) LE and the photo sensor(s) OS of the biosensor SE. That is, at least one pressure sensor PS, at least one light emitting unit LE and at least one photo sensor OS maybe disposed on a main portion MP, but not limited thereto. As shown in FIG. 3, a pressure sensor PS may for example include an electrode E3, an electrode E4 and a sensing layer MB located between the electrode E3 and the electrode E4, wherein the electrode E3 may be formed of the metal layer M4, and the electrode E4 may be formed of the metal layer M3 like the connecting element CT1, but not limited thereto. The sensing layer MB may include any suitable piezoelectric material, such as polyvinylidene fluoride (PVDF), but not limited thereto. According to the present embodiment, the on/off of the biosensors SE may for example be controlled through the pressure sensors PS. In detail, when a pressure sensor PS detects the pressure signal due to being pressed or deformed in other ways, the pressure sensor PS may turn on the biosensor SE which it belongs. On the other hand, when a pressure sensor PS does not detect the pressure signal, the pressure sensor PS may not turn on the biosensor SE which it belongs, or the off-state of the biosensor SE may be maintained. By controlling on/off of the biosensors SE through the pressure sensors PS, the power consumption of the biosensors SE maybe reduced, thereby improving the performance of the electronic device 200.

As mentioned above, the conductive line CW may electrically connect adjacent two of the biosensors SE. For example, the conductive line CW may be electrically connected to the driving units of these biosensors SE. In the present embodiment, in the process of electrically connecting two adjacent biosensors SE, the conductive line CW may be transferred to another layer. That is, the conductive line CW may be formed of different metal layers from the driving unit DU1 and/or the driving unit DU2, but not limited thereto. For example, as shown in FIG. 3, the conductive line CW of the present embodiment may be formed of the metal layer M1 and the metal layer M3, but not limited thereto. By transferring the conductive line CW to another layer, requirement of the space of the conductive line may be reduced, and the spatial configuration of the electronic device 200 may be improved.

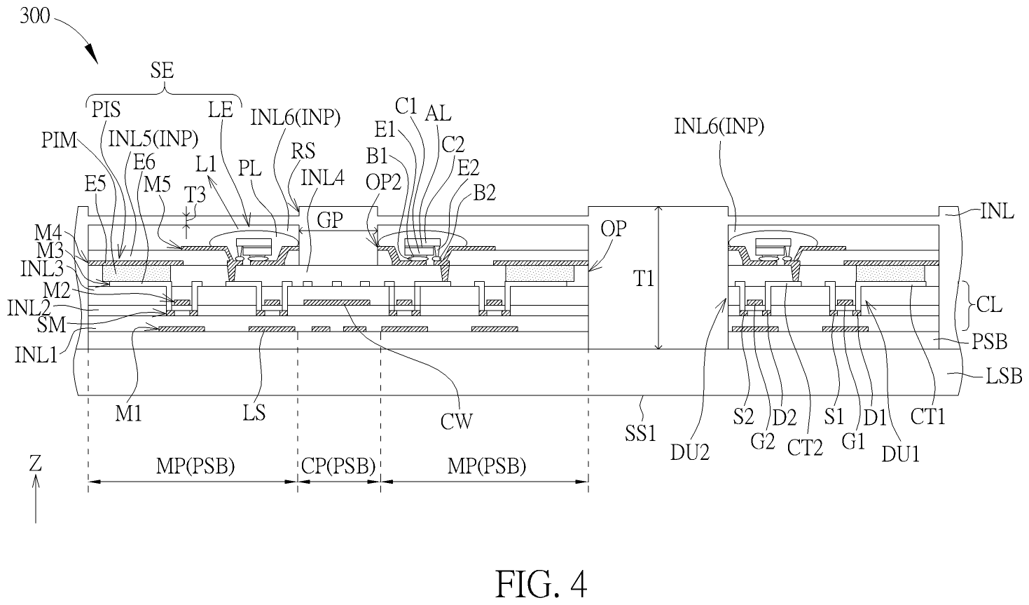

Referring to FIG. 4, FIG. 4 schematically illustrates a cross-sectional view of an electronic device according to a third embodiment of the present disclosure. According to the present embodiment, the biosensors SE of the electronic device 300 may include pressure sensors PIS and light emitting units LE, wherein a pressure sensor PIS may include an electrode E5, an electrode E6 and a sensing layer PIM located between the electrode E5 and the electrode E6. The electrode E5 may be formed of the metal layer M4, and the electrode E6 may for example be formed of the metal layer M3 like the connecting element CT2 electrically connected to the light emitting units LE, but not limited thereto. The sensing layer PIM may include any suitable piezoelectric material, such as polyvinylidene fluoride, but not limited thereto. Accordingly, the biosensors SE may for example be piezoelectric biosensors in the present embodiment, but not limited thereto. In the present embodiment, the light emitting units LE may for example provide the display function of the electronic device 300. For example, the result of detection or other information may be displayed, but not limited thereto. In some embodiments, when the electronic device does not have the function of light sensing and recognition, the biosensor SE may not include the light emitting unit LE.

In addition, the insulating layer INL of the electronic device 300 may be partially thinned in the present embodiment, thereby forming the insulating layer INL having an uneven upper surface, but not limited thereto. Specifically, as shown in FIG. 4, a thinning process may be performed on the portion of the insulating layer INL corresponding to the main portions MP to form recesses RS corresponding to the main portions MP. In this case, the portion of the insulating layer INL corresponding to the main portions MP may have a thickness T3, and the portion of the insulating layer INL corresponding to the connecting portions CP may have the thickness T1, wherein the thickness T3 may be lower than the thickness T1 in the present embodiment. That is, after the thinning process, the thickness of the insulating layer INL corresponding to the main portions MP may be lower than the thickness of the insulating layer INL corresponding to the connecting portions CP. Since the thickness T3 of the insulating layer INL corresponding to the main portions MP may be reduced through the thinning process, when the subject is in contact with the insulating layer INL, the distance between the biosensors SE and the subject may be lower. Therefore, the sensitivity or the accuracy of the biosensors SE may be improved. It should be noted that the shape of the insulating layer INL of the present embodiment is not limited to what is shown in FIG. 4. In some embodiments, the insulating layer INL may be disposed on the insulating layer INL6 and/or the supporting substrate LSB conformally, and the portion of the insulating layer INL corresponding to the main portions MP may be thinned through the thinning process, such that the thickness T3 of the insulating layer INL corresponding to the main portions MP may be lower than the thickness T1 of the portion of the insulating layer INL corresponding to the connecting portions CP.

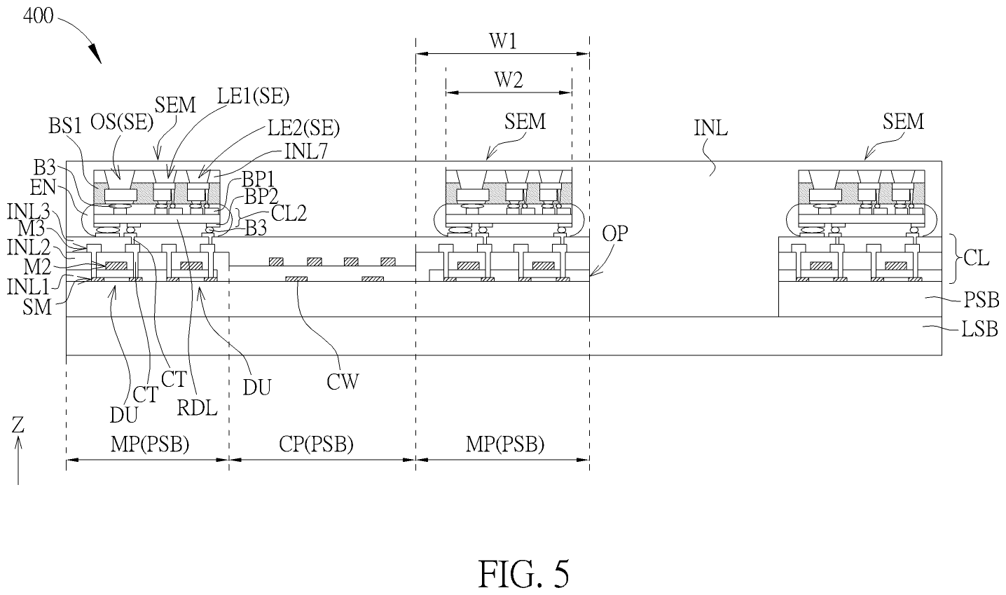

Referring to FIG. 5, FIG. 5 schematically illustrates a cross-sectional view of an electronic device according to a fourth embodiment of the present disclosure. According to the present embodiment, the electronic device 400 may include a plurality of biometric sensing modules SEM, and each of the biometric sensing modules SEM may be disposed corresponding to a main portion MP. Specifically, the elements (such as the photo sensor OS, the light emitting unit LE1 and the light emitting unit LE2) in the biosensor SE may be integrated in advance to form the biometric sensing module SEM, and then the biometric sensing module SEM may be transferred onto the circuit layer CL of the electronic device 400. The biometric sensing module SEM of the present embodiment may for example include the photo sensor OS, the light emitting unit LE1 and the light emitting unit LE2, but not limited thereto. The features of the photo sensor OS, the light emitting unit LE1 and the light emitting unit LE2 may refer to the content in the above-mentioned embodiments, and will not be redundantly described. In the present embodiment, the light emitting unit LE1 may for example emit visible light (that is, the light with a wavelength ranges from 340 nanometers (nm) to 700 nm), and the light emitting unit LE2 may for example emit far infrared light (that is, the light with a wavelength ranges from 15,000 nm to 1,000,000 nm), but not limited thereto. Since the biometric sensing module SEM may include different types of light emitting units, the light emitting unit to be used may be decided according to the demands of the user, thereby improving the convenience of the electronic device 400. It should be noted that the biometric sensing module SEM may further include a micro lens disposed on the photo sensor OS in some embodiments to improve the light receiving effect of the photo sensor OS, thereby improving the accuracy or the sensitivity of the biosensors SE. In addition, the biometric sensing module SEM may further include a light shielding structure BS1 that surrounds the photo sensor OS, the light emitting unit LE1 and the light emitting unit LE2, thereby reducing the influence of the stray light of the light emitting unit LE1 and the light emitting unit LE2 on the photo sensor OS. Furthermore, the biometric sensing module SEM may further include an insulating layer INL7 disposed on the photo sensor OS, the light emitting unit LE1 and the light emitting unit LE2, but not limited thereto.

The biometric sensing module SEM may further include a circuit layer CL2 in addition to the above-mentioned elements in the present embodiment, wherein the circuit layer CL2 may electrically connect the photo sensor OS, the light emitting unit LE1 and the light emitting unit LE2 to the driving units DU in the circuit layer CL. Specifically, as shown in FIG. 5, the circuit layer CL2 of the biometric sensing module SEM may for example include a plurality of bonding pads BP1, a plurality of bonding pads BP2 and a redistribution layer RDL located between the bonding pads BP1 and the bonding pads BP2, wherein the photo sensor OS, the light emitting unit LE1 and the light emitting unit LE2 may be electrically connected to the bonding pads BP1 through a bonding material B3, the bonding pads BP1 may be electrically connected to the bonding pads BP2 through the redistribution layer RDL, and the bonding pads BP2 may be electrically connected to the driving units DU in the circuit layer CL through the connecting elements CT, but not limited thereto. In the present embodiment, after the biometric sensing module SEM is bonded onto the circuit layer CL, a protecting layer EN may be disposed at a periphery of the circuit layer CL2, wherein the protecting layer EN may cover the circuit layer CL2 to provide the protecting effect of the circuit layer CL2 and fix the biometric sensing module SEM on the circuit layer CL. The protecting layer EN may include any suitable encapsulating material, fixing material or protecting material. For example, the protecting layer EN may include waterproof and oxygen-proof glue materials, but not limited thereto. According to the present embodiment, since the biosensors SE may be disposed on the circuit layer CL in the form of a module (that is, the biometric sensing module SEM), the layout of the wires in the circuit layer CL may be simplified or the number of the bonding pads may be reduced, thereby simplifying the bonding process of the biosensors SE. It should be noted that the circuit structures of the circuit layer CL and the circuit layer CL2 shown in FIG. 5 are merely exemplarily show the connection relationship between the elements, which are not the actual structures of the circuit layer CL and the circuit layer CL2.

In the present embodiment, as shown in FIG. 5, since the disposition of the biometric sensing module SEM may simplify the layout of the wires in the circuit layer CL, the design of the circuit layer CL corresponding to the connecting portions CP may be simplified. For example, as shown in FIG. 5, the portion of the circuit layer CL corresponding to the connecting portions CP may not include the insulating layer INL1, and the conductive line CW may for example be formed of the metal layer M2 (or the metal layer M3), but not limited thereto.

According to the present embodiment, the width of the biometric sensing module SEM in a direction parallel to the surface of the supporting substrate LSB (for example, the direction X or the direction Y, the direction Y is taken as an example in FIG. 5, but not limited thereto) may be less than the width of a main portion MP in the direction. For example, as shown in FIG. 5, the biometric sensing module SEM has a width W2 in the direction Y parallel to the surface of the supporting substrate LSB, and the main portion MP has a width W1 in the direction Y, wherein the width W2 may be lower than the width W1. In other words, the projected area of the biometric sensing module SEM on a plane parallel to the surface of the supporting substrate LSB (for example, the plane X-Y) may be lower than the projected area of the main portion MP on the same plane. Accordingly, before the biometric sensing module SEM is transferred onto the circuit layer CL, the width W1 of the main portion MP may be confirmed at first, and the width W2 of the biometric sensing module SEM may be adjusted accordingly, but not limited thereto. According to the present embodiment, by making the width W2 of the biometric sensing module SEM lower than the width W1 of the main portion MP, the biometric sensing module SEM may not protrude from the main portion MP in a top view direction (the direction Z). Therefore, the possibility that the biometric sensing module SEM is skewed due to instability may be reduced, thereby improving the reliability of the electronic device 400.

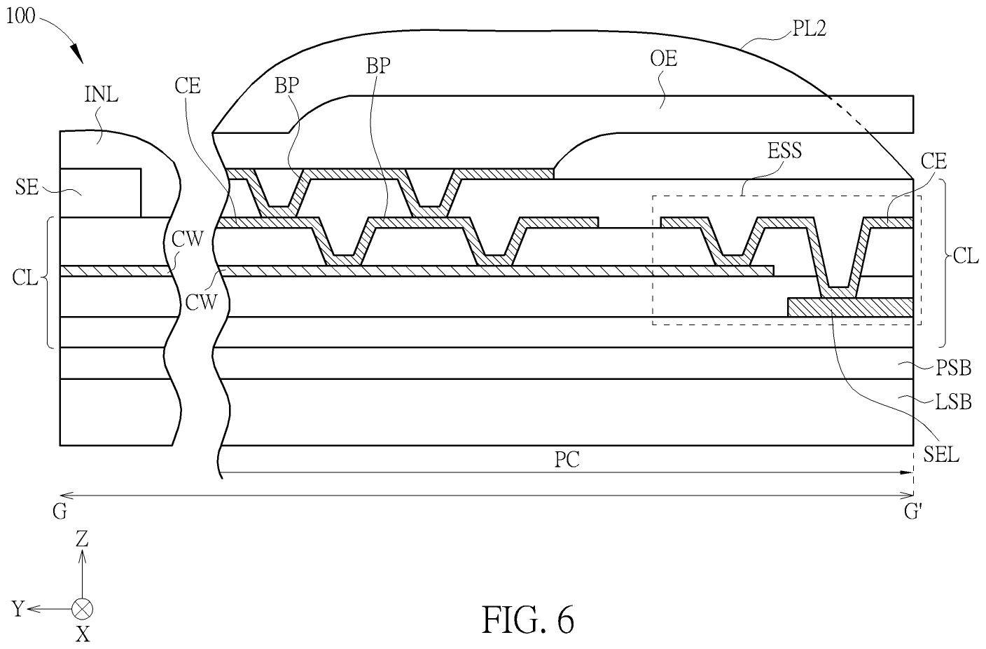

Referring to FIG. 6 as well as FIG. 1, FIG. 6 schematically illustrates a cross-sectional view of the electronic device shown in FIG. 1 along a cutting line G-G′. According to the present embodiment, as mentioned above, the signal lines (such as the conductive line CW, but not limited thereto) in the circuit layer CL may be electrically connected to the bonding pads BP in the peripheral circuit region PC and electrically connected to the external electronic element OE through the bonding pads BP. In detail, as shown in FIG. 6, the peripheral circuit region PC of the electronic device 100 may include the bonding pads BP, wherein the bonding pads BP may be formed by at least one metal layer (for example, two metal layers, but not limited thereto). The conductive line CW may extend to the peripheral circuit region PC and be in contact with the bonding pads BP, and the bonding pads BP may be electrically connected to the external electronic element OE, thereby electrically connecting the signal lines in the circuit layer CL to the external electronic element OE. It should be noted that the circuit structure in the peripheral circuit region PC of the electronic device 100 is not limited to what is shown in FIG. 6, and other suitable circuit structures may be included in the peripheral circuit region PC. In addition, the electronic device 100 may further include a protecting layer PL2 disposed on the bonding pads BP and covering at least the junction between the external electronic element OE and the bonding pads BP, so as to provide the protecting effect, but not limited thereto.

In the present embodiment, the electronic device 100 may optionally include an electrostatic discharge (ESD) protecting element ESS (as shown in FIG. 1 and FIG. 6), wherein the ESD protecting element ESS may for example be disposed in the peripheral circuit region PC, but not limited thereto. As shown in FIG. 6, the ESD protecting element ESS may for example include a connecting element CE and a conductive layer SEL, wherein the bonding pads BP and the conductive wire CW may be electrically connected to the connecting element CE, and the connecting element CE may be electrically connected to the conductive layer SEL. That is, the bonding pads BP and the conductive line CW may be electrically connected to the conductive layer SEL through the connecting element CE. The conductive layer SEL may for example include a semiconductor layer, but not limited thereto. According to the present embodiment, the ESD protecting element ESS may discharge the static electricity accumulated on the bonding pads BP and/or the conductive line CW, or prevent the static charge from accumulating on the bonding pads BP and/or the conductive line CW. Therefore, the possibility of damage of the electronic elements in the electronic device 100 due to electrostatic discharge may be reduced. It should be noted that although it is not shown in the figure, other regions of the electronic device 100 may include the ESD protecting element ESD, but the present disclosure is not limited thereto.

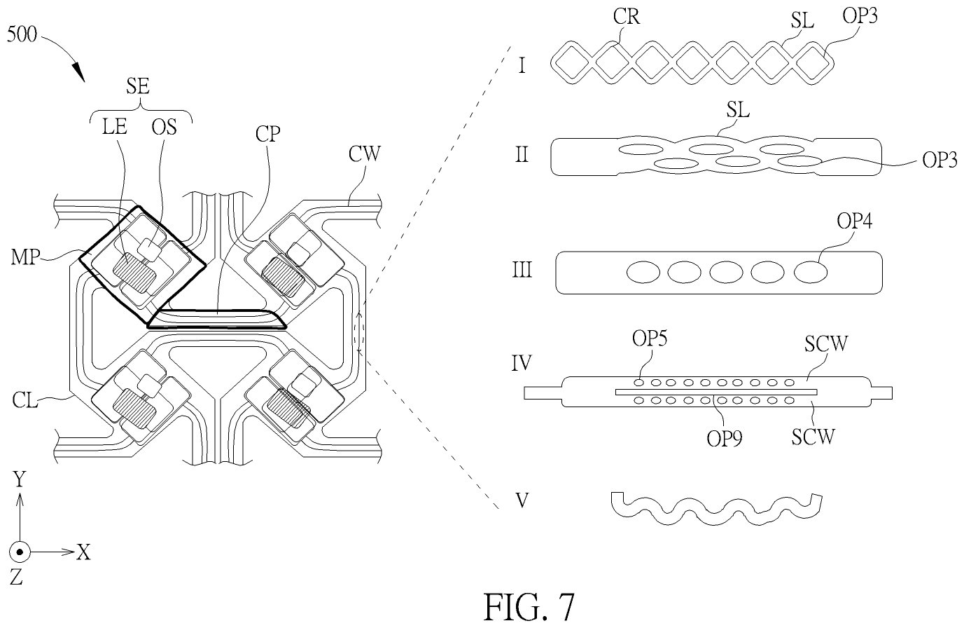

Referring to FIG. 7, FIG. 7 schematically illustrates a top view of a conductive line of an electronic device according to a fifth embodiment of the present disclosure. In order to simplify the figure, FIG. 7 just exemplarily shows the circuit layer CL, the conductive line CW and the biosensors SE of the electronic device 500, and other elements and/or layers are omitted. As shown in FIG. 7, compared with the electronic device 100 in the first embodiment, the arrangement of the main portions MP and the connecting portions CP may be different in the present embodiment, thereby forming the circuit layer CL (or the patterned substrate PSB) with a different pattern. That is, the electronic device of the present disclosure may include the circuit layer CL or the patterned substrate PSB with any suitable pattern according to the demands of the design of the product.

According to the present embodiment, the conductive line(s) CW in the circuit layer CL maybe disposed on the connecting portions CP and extend on the connecting portions CP. Since the connecting portions CP may be deformed in a greater degree when the electronic device 500 is stretched or deformed in other ways, the conductive line(s) CW disposed on the connecting portions CP may be designed to reduce the possibility of breaking or damage of the conductive line CW due to the stress generated by deformation. In addition, the conductive line CW of the present embodiment may for example include conductive materials, silver wires, metal nanoparticles, metal nanowires, carbon nanotubes, conductive polymers, other suitable materials or combinations of the above-mentioned materials, so as to improve the bending resistance of the conductive line CW, but not limited thereto. FIG. 7 shows some examples of the conductive line CW of the present embodiment. In the example I and the example II, the conductive line CW may include openings such as openings OP3, and at least a portion of the side SL of the conductive line CW may be wavy-shaped. The difference between the example I and the example II is that the two ends of the conductive line CW in the example I may include the openings OP3, and the two ends of the conductive line CW in the example II do not include the opening OP3. In addition, the inner edge of the opening OP3 of the conductive line CW in the example I may have a turning portion CR, wherein the turning portion CR may be curved or arc-shaped, and the opening OP3 of the conductive line CW in the example II may be curved or arc-shaped, but not limited thereto. In the example III, the side of the conductive line CW may be linear, and the conductive line CW may include a plurality of openings OP4. In the example IV, at least a portion of the conductive line CW (for example, the portion of the conductive line CW on the connecting portions CP) may be divided into two sub conductive lines SCW by an opening OP9, wherein the sub conductive line SCW may include a plurality of openings OP5. In some embodiments, the sub conductive line SCW may not include the opening OP5. In the example V, the conductive line CW itself may substantially be wavy-shaped or include any suitable curved shape. Through the designs of the conductive lines CW mentioned above, the bending resistance of the conductive line CW may be improved, thereby reducing the possibility of breaking of the conductive line CW disposed on the connecting portions CP due to stretching or other deformation. The features such as material, shape, and the like of the conductive line CW in the present embodiment may be applied to each of the embodiments of the present disclosure. In addition, the shape of the conductive line CW of the present embodiment is not limited to the shapes in the above-mentioned examples, which may further include other suitable shapes.

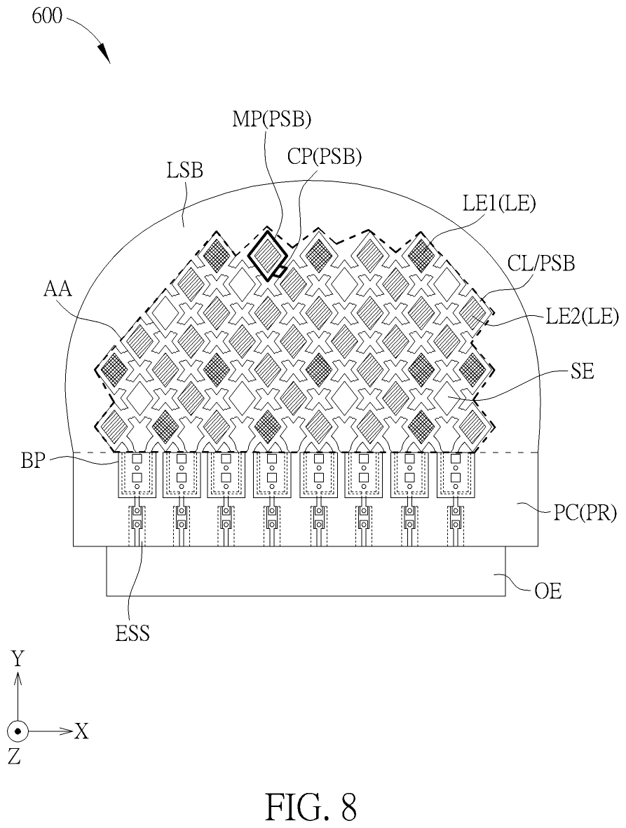

Referring to FIG. 8, FIG. 8 schematically illustrates a top view of an electronic device according to a sixth embodiment of the present disclosure. In order to simplify the figure, the fan out region FO and the dummy region DUM of the electronic device 600 are omitted in FIG. 8. In addition, the patterns of the circuit layer CL and/or the patterned substrate PSB of the electronic device 600 shown in FIG. 8 may be different from the patterns of the circuit layer CL and/or the patterned substrate PSB of the electronic device 100 and the electronic device 500 mentioned above, but not limited thereto. According to the present embodiment, various kinds of elements may be disposed on the main portions MP of the electronic device 600, so as to provide various functions of the electronic device 600. For example, in the electronic device 600, the light emitting units LE may be disposed on a portion of the main portions MP, and the biosensors SE may be disposed on another portion of the main portions MP, wherein the light emitting units LE may include the light emitting units LE1 that emit visible light (such as red light) and the light emitting units LE2 that emit far infrared light, but not limited thereto. That is, the light emitting units LE1, the light emitting units LE2 and the biosensors SE may respectively be disposed on the main portions MP of the electronic device 600, but not limited thereto. The biosensors SE here may be the biosensor SE in anyone of the embodiments mentioned above. In addition, different types of biosensors SE may be included on the main portions MP of the electronic device 600, but not limited thereto. When the biosensor SE is a photoelectric biosensor, the biosensor SE may include the light emitting unit LE to provide a light source for detection, but not limited thereto. In some embodiments, the biosensor SE may not include the light emitting unit LE, and the light source for detection may be provided by the light emitting units LE on other main portions MP adjacent to the biosensor SE, but not limited thereto. According to the present embodiment, since the electronic elements providing functions such as illumination, sensing, and the like may be disposed on different main portions MP of the electronic device 600, the effect of integration of diagnosis and therapy may thereby be achieved. Specifically, the biosensors SE may be used to detect the physiological information of the subject, and the light emitting units LE1 and/or the light emitting units LE2 may be used for treatment (such as light therapy) according to the result of detection. For example, when the subject is injured, the biosensors SE may be used to detect the condition of the wound and obtain information such as wound image, pH value of wound, oxygen saturation, and the like. After that, the light emitting units LE1 and/or the light emitting units LE2 may be used to apply light therapy to the wound according to the result of detection, wherein the type of the light emitting units LE (or the wavelength of the light) used for therapy may be determined according to the result of detection of the biosensors SE. In the present embodiment, the light emitting units LE may for example perform light therapy through pulse lighting, so as to reduce the time for the tissue of the wound to receive heat through light, thereby reducing the possibility of damage to the tissue of the wound due to excess heat. In addition, the intense pulsed light with specific frequency may resonate with biological signals to maximize biological effects. For example, the intense pulsed light capable of healing the wound may for example have a frequency of less than 100 hertz (Hz), so the light emitting units LE1 and/or the light emitting units LE2 may for example emit intense pulsed light having a frequency of less than 100 Hz, but not limited thereto. It should be noted that the types of electronic elements included in the electronic device 600 are not limited to the above-mentioned content, and electronic elements with various functions may be included in the electronic device 600 according to the demands of the design of the product.

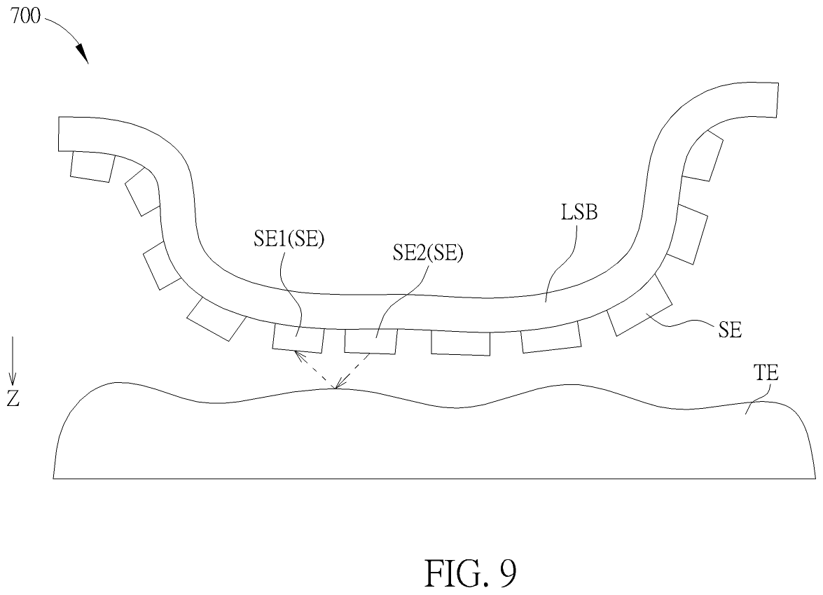

Referring to FIG. 9, FIG. 9 schematically illustrates an application of an electronic device according to a seventh embodiment of the present disclosure. In order to simplify the figure, FIG. 9 just exemplarily shows the supporting substrate LSB and the biosensors SE disposed on the supporting substrate LSB of the electronic device 700, and the layers such as the circuit layer CL, the patterned substrate PSB, and the like are omitted. In addition, the biosensor SE is exemplarily shown as a single layer in FIG. 9, and the detailed structure of the biosensor SE may refer to the content in the above-mentioned embodiments, which will not be redundantly described. As mentioned above, when the electronic device 700 is used to obtain the physiological information of the subject, a signal (such as a light signal) may be transmitted from a signal transmitting source (such as the light emitting units LE) at first, and the signal may be reflected by the subject, received by a signal receiving source (such as the photo sensor OS), and converted into physiological information. According to the present embodiment, when transmitting and receiving the light signals, the signal receiving source may choose to receive the signals from the signal transmitting source with approximately the same Gaussian curvature as the signal receiving source. In other words, the photo sensor OS in a biosensor SE may receive the light signals emitted by the light emitting units LE in other biosensors SE having the same Gaussian curvature as the biosensor SE or receive the light signals emitted by the light emitting unit LE in the biosensor SE. For example, when the shape of the electronic device 700 in operation is as shown in FIG. 9, the Gaussian curvature of the biosensor SE1 and the Gaussian curvature of the biosensor SE2 may approximately be the same. In this case, the photo sensor (not shown) in biosensor SE1 may receive the light signals emitted from the light emitting units (not shown) in biosensor SE2 or the light signals emitted from the light emitting units (not shown) in biosensor SE1, but not limited thereto. By making a signal receiving source receive the signal emitted from the signal transmitting source with substantially the same Gaussian curvature as the signal receiving source, the influence of stray light on the signal receiving source may be reduced, thereby improving the accuracy or the sensitivity of the biosensors SE.

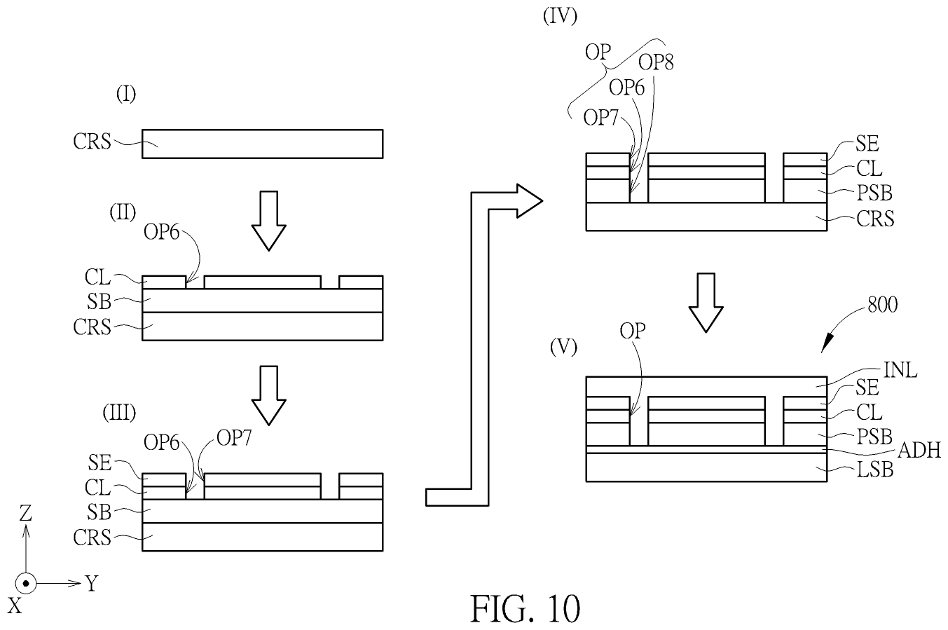

Referring to FIG. 10, FIG. 10 schematically illustrates the manufacturing process of an electronic device according to an eighth embodiment of the present disclosure. According to the present embodiment, the manufacturing method of the electronic device 800 may include the following steps. First, as shown in the step (I), a carrier substrate CRS may be provided, wherein the carrier substrate CRS may for example include a rigid substrate. The material of the rigid substrate may for example include glass, quartz, sapphire, ceramic, other suitable materials or combinations of the above-mentioned materials. Then, as shown in the step (II), the substrate SB may be formed on the carrier substrate CRS, and the circuit layer CL maybe formed on the substrate SB. After the circuit layer CL is formed, the openings OP6 may be formed in the circuit layer CL to pattern the circuit layer CL. It should be noted that the openings OP6 may have any suitable shape or inclined surfaces in the cross-sectional view shown in FIG. 10, but the present disclosure is not limited thereto. After that, as shown in the step (III), the patterned biosensors SE may be disposed on the circuit layer CL. The biosensor SE including detailed circuits and electrical elements is represented as a simplified patterned layer here. Specifically, a sensor layer including a plurality of biosensors SE maybe disposed on the circuit layer CL at first, and then the openings OP7 may be formed in the sensor layer, thereby patterning the sensor layer and forming the biosensors SE corresponding to the main portions MP, but not limited thereto. In some other embodiments, after the patterned circuit layer CL is formed, the unit (s) including the plurality of biosensors SE may be transferred onto the circuit layer CL, and there is no need to dispose opening in the biosensor SE. As shown in the step (III) of FIG. 10, since the pattern of the sensor layer including the biosensors SE may be the same as the pattern of the circuit layer CL, the openings OP6 may overlap the openings OP7 in the direction Z, but not limited thereto. After the patterned biosensors SE are formed, the step (IV) may be performed to form the openings OP8 in the substrate SB, so as to pattern the substrate SB to form the patterned substrate PSB mentioned above. Since the pattern of the patterned substrate PSB may for example be the same as the pattern of the circuit layer CL and the pattern of the sensor layer, the openings OP8 may overlap the openings OP6 and the openings OP7 in the direction Z, wherein the openings OP6, the openings OP7 and the openings OP8 may form the openings OP shown in FIG. 2. After the patterned substrate PSB is formed, the step (V) may be performed to remove the carrier substrate CRS and adhere the supporting substrate LSB to the patterned substrate PSB through an adhesive layer ADH. After that, the insulating layer INL covering the biosensors SE and filled into the openings OP may be disposed, thereby forming the electronic device 800. According to the present embodiment, after the electronic device 800 is formed, the circuit layer CL or the biosensors SE may for example be located at the neutral axis of the electronic device 800, thereby reducing the influence of stress on the circuit layer CL or the biosensors SE. It should be noted that although the openings OP6 in the circuit layer CL are formed at first, and then the openings OP7 in the sensor layer are formed in the manufacturing method of the electronic device 800 in the present embodiment, the present disclosure is not limited thereto. In some embodiments, after the circuit layer CL is formed, the biosensors SE maybe disposed at first, and the openings maybe formed in the circuit layer CL and the sensor layer at the same time. The manufacturing method of the electronic device 800 of the present embodiment may be applied to each of the embodiments of the present disclosure.

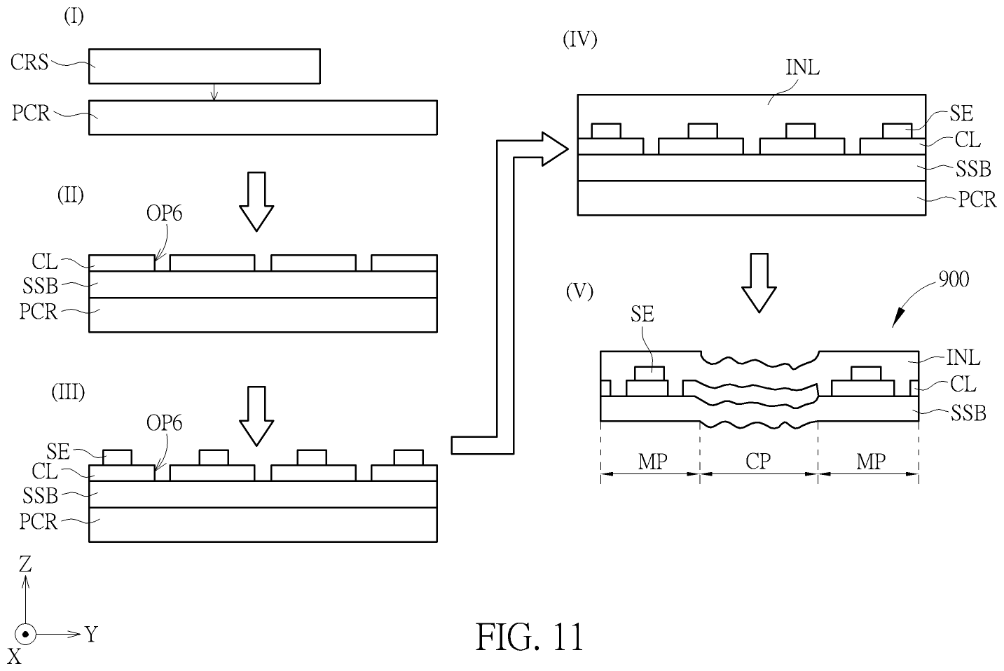

Referring to FIG. 11, FIG. 11 schematically illustrates the manufacturing process of an electronic device according to a ninth embodiment of the present disclosure. According to the present embodiment, the manufacturing method of the electronic device 900 may include the following steps. First, as shown in the step (I), a carrier substrate CRS may be provided, wherein the carrier substrate CRS may be a flexible substrate. For example, the carrier substrate CRS may be a rubber substrate, but not limited thereto. After the carrier substrate CRS is provided, a pre-stretch step may be performed on the carrier substrate CRS to stretch the carrier substrate CRS to form a pre-stretched carrier substrate PCR, and the stretched state of the pre-stretched carrier substrate PCR is maintained. Then, as shown in the step (II), a stretchable substrate SSB may be formed on the pre-stretched carrier substrate PCR, and the patterned circuit layer CL may be formed on the stretchable substrate SSB. Specifically, a complete circuit layer CL may be formed on the stretchable substrate SSB at first, and the openings OP6 maybe formed in the circuit layer CL to pattern the circuit layer CL. The material of the stretchable substrate SSB may refer to the materials of the patterned substrate PSB mentioned above, and will not be redundantly described. As shown in the step (III), after the patterned circuit layer CL is formed, the biosensors SE may be disposed on the circuit layer CL, wherein the biosensors SE may for example be transferred onto the circuit layer CL, but not limited thereto. After that, the step (IV) may be performed to dispose the insulating layer INL covering the biosensors SE and filled into the openings OP. After the insulating layer INL is disposed, the step (V) may be performed to remove the pre-stretched carrier substrate PCR, thereby forming the electronic device 900. Since the pre-stretched carrier substrate PCR is in a stretched state, after the pre-stretched carrier substrate PCR is removed, at least a portion of the stretchable substrate SSB, the circuit layer CL and the insulating layer INL would shrink inward and may be stretched or deformed more easily (as shown in the step (V)). According to the present embodiment, the portion of the stretchable substrate SSB that can be stretched or deformed more easily may be defined as the connecting portion CP, wherein the connecting portion CP may connect adjacent two of the main portions MP, but not limited thereto. Through the manufacturing method of the electronic device 900 of the present embodiment, a stretchable biometric sensing device may be formed.

In summary, an electronic device is provided by the present disclosure, wherein the electronic device may include biosensors and may serve as biometric sensing device. Since the electronic device may include the biocompatible covering layer that covers the biosensors, the influence of the electronic elements in the electronic device on the users may be reduced, or the possibility of damage to the electronic elements in the electronic device may be reduced. In addition, since the electronic device of the present disclosure is flexible, the adaptability of electronic device in the using environment may be improved.

Those skilled in the art will readily observe that numerous modifications and alterations of the device and method may be made while retaining the teachings of the disclosure. Accordingly, the above disclosure should be construed as limited only by the metes and bounds of the appended claims.

What is claimed is:

1. An electronic device, comprising:
a patterned substrate having a plurality of main portions and a plurality of connecting portions, wherein at least one of the plurality of connecting portions connects two adjacent ones of the plurality of main portions;
a plurality of biosensors disposed corresponding to the plurality of main portions;
a conductive line disposed on the at least one of the plurality of connecting portions and electrically connecting two adjacent ones of the plurality of biosensors; and
an insulating layer disposed on the plurality of biosensors and the conductive line.

2. The electronic device of claim 1, further comprising a plurality of insulating patterns disposed on the plurality of biosensors respectively, wherein two adjacent ones of the plurality of insulating patterns are spaced apart by a gap overlapping one of the plurality of connecting portions.

3. The electronic device of claim 1, further comprising a supporting substrate disposed under the patterned substrate.

4. The electronic device of claim 1, wherein the conductive line has a plurality of openings.

5. The electronic device of claim 1, wherein the plurality of biosensors and the conductive line are disposed on a same side of the patterned substrate.

6. The electronic device of claim 1, wherein the plurality of biosensors comprises a plurality of light emitting units and a plurality of photo sensors, the plurality of light emitting units are respectively disposed corresponding to the plurality of main portions and configured to emit a light, and the plurality of photo sensors are configured to sense the light.

7. The electronic device of claim 6, further comprising a light shielding layer disposed on the plurality of biosensors and having a plurality of through holes overlapped with the plurality of biosensors respectively.

8. The electronic device of claim 1, wherein the plurality of biosensors comprise piezoelectric materials.
